(19) United States
(12) Patent Application Publication
(10) Pub. No.: US 2010/0048753 A1
(43) Pub. Date: Feb. 25, 2010

(54) ALDEHYDE AND ALCOHOL COMPOSITIONS DERIVED FROM SEED OILS

(75) Inventors: Wei-Jun Peng, Midland, MI (US); David A. Babb, Lake Jackson, TX (US); Aaron W. Sanders, Missouri City, TX (US); Christopher W. Derstine, Midland, MI (US); Jorge Jimenez, Lake Jackson, TX (US); Zenon Lysenko, Midland, MI (US); Kurt D. Olson, Freeland, MI (US); Joe D. Phillips, Lake Jackson, TX (US); Brian M. Roesch, Middletown, DE (US); Alan K. Schrock, Lake Jackson, TX (US)

Correspondence Address: The Dow Chemical Company, Intellectual Property Section, P.O. Box 1967, Midland, MI, 48641-1967, US

(73) Assignee: Dow Global Technologies Inc., Midland, MI (US)

(21) Appl. No.: 12/518,432

(22) PCT Filed: Dec. 3, 2007
(86) PCT No.: PCT/US2007/086222

§ 371 Date: Oct. 30, 2009

Related U.S. Application Data
(60) Provisional application No. 60/874,213, filed on Dec. 11, 2006.

(51) Int. Cl. C08G 18/00; C07C 53/00; C07C 69/34

(52) U.S. Cl. 521/172; 528/80; 554/227; 560/198

(57) ABSTRACT

An aldehyde composition containing a mixture of mono-formyl-, diformyl-, and triformyl-substituted fatty acids and/or fatty acid esters having a di-aldehyde/tri-aldehyde weight ratio of less than 5/1 and an average functionality number from greater than 0.96 to less than 1.26. A monomer alcohol composition containing a mixture of mono-hydroxymethyl-, dihydroxymethyl-, and trihydroxymethyl-substituted fatty acids and/or fatty acid esters having a diol/triol weight ratio of less than 5/1 and an average functionality number from greater than 0.90 to less than 1.20. The monomer alcohol can be converted into an oligomeric polyol for use in the manufacture of polyurethane flexible foams.

### CROSS-REFERENCE TO RELATED APPLICATION

This application claims the benefit of U.S. Provisional Patent Application 60/874,213, filed Dec. 11, 2006.

### BACKGROUND OF THE INVENTION

In one aspect, this invention pertains to an aldehyde composition prepared by hydroformylation of one or more unsaturated fatty acids or unsaturated fatty acid esters derived from seed oils. In another aspect, this invention pertains to an alcohol composition prepared by hydrogenation of the aforementioned aldehyde composition.

At the present time, industry-wide efforts are underway to replace, where possible, petroleum-based chemical feedstocks with non-petroleum-based chemical feedstocks. Seed oils, which comprise a mixture of saturated and unsaturated fatty acid esters, provide a promising source of renewable non-petroleum-based feedstocks for industrial utilization. Aldehydes can be derived from transesterification and hydroformylation of seed oils. The aldehydes obtained therefrom can be converted via hydrogenation into alcohols, which in turn can be used as monomer feedstocks for conversion into polyols that find use in the manufacture of polyurethanes. Aldehydes derived from seed oils can also be converted into polyamines, carboxylic acids, hydroxy acids, amino alcohols, amino acids, and other commercially useful derivatives.

In order to be useful in present day polyurethane manufacture, non-petroleum-based polyols should provide similar reactivity and similar urethane end-products at acceptable cost, as compared with conventional petroleum-based polyols. Inasmuch as the properties of polyurethanes are known to vary with the specific polyol composition employed, non-petroleum-based polyols may also offer opportunities for preparing unconventional polyurethane products with novel properties. Whatever the desired outcome, non-petroleum-based aldehyde and alcohol monomer compositions should be engineered such that the polyols derived therefrom yield polyurethanes of acceptable properties for the desired end-use. Polyols used in the manufacture of polyurethane flexible foams, for example, should provide for acceptable cross-link density, that is, a cross-link density neither too high nor too low; else the foam has unacceptable rigidity or flexibility. The invention described herein pertains particularly to aldehyde and alcohol monomer compositions derived from seed oils, which provide for polyols having acceptable properties for the manufacture of polyurethane flexible foams.

Prior art, exemplified by U.S. Pat. No. 3,787,459, disclose a process for converting unsaturated vegetable oil materials via hydroformylation into formyl (aldehyde) products. Disclosed vegetable oils include soybean, linseed, and safflower oils, and their derivatives. As best as can be determined, the composition disclosed in U.S. Pat. No. 3,787,459 consists of from 24 to 92 percent monoformyl product, and when diformyl product is present, from 17 to 75 percent diformyl, by weight, based on the total weight of the composition.

Other prior art, such as EP-B1-711748, disclose a process for preparing di- and polyformylcarboxylic esters by hydroformylation of esters of multiply unsaturated fatty acids, such as soybean oil, sunflower oil, and linseed oil. The resulting aldehyde composition, as illustrated in the examples, appears to comprise from 23 to 35 percent monoformyl, from 12 to 31 percent diformyl, and from 3 to 29 percent triformyl products, by weight, based on the total weight of the composition.

Yet other prior art, illustrated in U.S. Pat. No. 5,177,228, disclose the hydroformylation of a single unsaturated fatty acid ester, such as methyl oleate, to a single product monoformyl fatty acid ester, such as, methylformyl stearate.

WO 2004/096744 discloses an aldehyde composition derived from seed oils comprising a mixture of formyl-substituted fatty acids or fatty acid esters comprising in terms of formyl distribution from greater than about 10 to less than about 95 percent monoformyl, from greater than about 1 to less than about 65 percent diformyl, and from greater than about 0.1 to less than about 10 percent triformyl, by weight, based on the total weight of the composition, further characterized by a diformyl to triformyl weight ratio of greater than 5/1. WO 2004/096744 also discloses an alcohol composition comprising a mixture of hydroxymethyl-substituted fatty acids or fatty acid esters comprising in terms of hydroxy distribution from greater than about 10 to less than about 95 percent monoalcohol, from greater than about 1 to less than about 65 percent diol, and from greater than about 0.1 to less than about 10 percent triol, by weight, based on the total weight of the composition, further characterized as having a diol to triol weight ratio greater than 5/1. In practice, the disclosed compositions are limited to derivatives of soy oils and oils similar to soy, which contain a large amount of di-unsaturated fatty acids and/or fatty acid esters, for example, greater than about 50 weight percent, and a low amount of tri-unsaturated fatty acids and/or fatty acid esters, for example, less than about 10 weight percent. In contrast, feedstocks having lower quantities of di-unsaturates and higher quantities of tri-unsaturates cannot supply products having a functional di/tri weight ratio greater than 5/1.

One difficulty in using a feedstock derived from seed oils is that the composition of the feedstock varies significantly from one seed oil to another, making it difficult to predict an aldehyde or alcohol monomer composition for use in polymer applications, such as, flexible polyurethane foams. The prior art typically uses “percent conversion” to describe the degree of conversion of a seed oil in a functionalization process, such as, hydroformylation, where “conversion” is typically defined as consumption of olefin molecules. Although “percent conversion” serves well in single component petroleum based feedstocks, “percent conversion” is inadequate in describing the degree of functionalization in seed oil-based feedstocks containing a mixture of compounds having none, one, two, or three olefinic bonds per molecule.

In view of the above, a need exists in the art for aldehyde and alcohol monomer compositions derived from renewable, non-petroleum-based seed oil feedstocks that have compositions in terms of mono-, di-, and tri-unsaturated components significantly different from soybean oil. Moreover, a need exists to employ such aldehyde and monomer alcohol compositions to produce polyols having acceptable properties for use in polymer applications, specifically, polyurethane flexible foams. A need also exists for a method of preparing aldehyde and alcohol monomer compositions of predictable composition independent of the seed oil source.

### SUMMARY OF THE INVENTION

In a first aspect, this invention provides for a novel aldehyde composition comprising a mixture of formyl-substituted fatty acids and/or fatty acid esters, which comprises in terms of formyl distribution from greater than about 30 to less than about 95 percent mono-aldehyde, from greater than about 0.4 to less than about 37 percent di-aldehyde, and from greater than about 0.1 to less than about 34 percent tri-aldehyde, by weight, based on the total weight of the composition. In addition, the aldehyde composition of this invention is characterized by a di-aldehyde to tri-aldehyde (di-al/tri-al) weight ratio of less than 5/1 and an average functionality number ranging from greater than 0.96 to less than 1.26. The term “average functionality number” and its determination are explained in detail hereinafter.

The novel aldehyde composition of this invention can be hydrogenated or hydroaminated to the corresponding alcohol or amine, which provides for a useful monomer in the preparation of polyols or polyamines, respectively.

In a second aspect, this invention provides for a novel alcohol composition comprising a mixture of hydroxymethyl-substituted fatty acids and/or fatty acid esters, which comprises in terms of hydroxy distribution from greater than about 30 to less than about 90 percent monoalcohol, from greater than about 0.4 to less than about 34 percent dialcohol, and from greater than about 0.1 to less than about 31 percent trialcohol, by weight, based on the total weight of the composition. In addition, the novel alcohol composition of this invention has a dialcohol to trialcohol (diol/triol) weight ratio less than 5/1 and an average functionality number ranging from greater than 0.90 to less than 1.20.

The novel alcohol composition is useful as a monomer in the preparation of polyols, which finds utility in polymer applications including polyurethane flexible foams and other polyurethane products.

In a third aspect, this invention pertains to a polyester polyol composition comprising a reaction product of an alcohol composition with an initiator compound having from 2 to 8 hydroxyl groups per molecule and a molecular weight of about 90 to about 6000, the alcohol composition comprising a mixture of hydroxymethyl-substituted fatty acids and/or fatty acid esters, which comprises in terms of hydroxy distribution from greater than about 30 to less than about 90 percent monoalcohol, from greater than about 0.4 to less than about 34 percent dialcohol, and from greater than about 0.1 to less than about 31 percent trialcohol, by weight, based on the total weight of the alcohol composition, and having a dialcohol to trialcohol (diol/triol) weight ratio less than 5/1 and an average functionality number ranging from greater than 0.90 to less than 1.20.

In a fourth aspect, this invention pertains to a polyurethane comprising a reaction product of the aforementioned polyester polyol composition with at least one polyisocyanate.

### DRAWINGS

FIG. 1 illustrates impurity compounds that may be found in the alcohol composition including lactol, lactone, saturated cyclic ether, and unsaturated cyclic ether.

FIG. 2 illustrates additional impurity compounds that may be found in the alcohol composition including dimer and condensation heavies.

### DETAILED DESCRIPTION OF THE INVENTION

The inventions described herein allow for beneficial exploitation of renewable, naturally occurring and genetically modified seed oils in the preparation of non-petroleum-based aldehyde and alcohol monomer feedstocks useful in the manufacture of industrial chemicals, preferably, polyurethanes. In a first aspect, this invention provides for a novel aldehyde composition comprising a mixture of formyl-substituted fatty acids and/or fatty acid esters comprising in terms of formyl distribution from greater than about 30 to less than about 95 percent monoaldehyde, from greater than about 0.4 to less than about 37 percent di-aldehyde, and from greater than about 0.1 to less than about 34 percent tri-aldehyde, by weight, based on the total weight of the composition. The aldehyde composition is further characterized as comprising a di-aldehyde/tri-aldehyde (di-al/tri-al) weight ratio of less than 5/1 and an average functionality number ranging from greater than 0.96 to less than 1.26.

For the purposes of this invention, the term “monoaldehyde” (or “mono-al”) refers to any fatty acid or fatty acid ester having one formyl (—CHO) substituent per molecule. The formyl substituent may occur at any saturated carbon atom along the fatty acid chain, which may be fully saturated or may additionally contain one or more unsaturated C═C double bonds. The unsaturated C═C double bonds are those that were present in the seed oil, but which remained unconverted in the process (hydroformylation) of producing the monoaldehyde. Analogously, the terms “di-aldehyde” (“di-al) and “tri-aldehyde” (“tri-al”) refer herein to any fatty acid or fatty acid ester having two or three formyl substituents, respectively, per molecule, such substituents being distributed among the saturated carbon atoms along the fatty acid chain. Likewise, the fatty acid or fatty acid ester chain of the di-aldehyde and tri-aldehyde may be fully saturated or may additionally contain one or more unsaturated C═C double bonds, although unsaturated triformyl compounds may be less likely to occur. Furthermore, the words “mono-aldehyde,” “di-aldehyde,”and “tri-aldehyde” each individually include single species thereof or mixtures of such species differentiated by fatty acid chains having different lengths. As an example, the term “mono-aldehyde” can refer to a single species of C₁₆ mono-aldehyde as well as a mixture of C₁₆ and C₁₈ mono-aldehydes.

As applied to the novel aldehyde composition, the term “average functionality number” is defined as the average number of aldehyde (formyl) functionality per fatty acid or fatty acid ester chain, as explained in further detail hereinafter.

In a preferred embodiment, the aldehyde composition comprises greater than about 40 percent, more preferably, greater than about 50 percent mono-aldehyde, that is, mono-formyl-substituted fatty acids or fatty acid esters, by weight, based on the total weight of the aldehyde composition. In a preferred embodiment, the aldehyde composition comprises less than about 93 percent, and more preferably, less than about 90 percent mono-aldehyde, by weight. In another preferred embodiment, the aldehyde composition comprises greater than about 1 percent, more preferably, greater than about 2 percent di-aldehyde, that is, diformyl-substituted fatty acids or fatty acid esters, by weight. In another preferred embodiment, the aldehyde composition comprises less than about 32 percent, more preferably, less than about 27 percent di-aldehyde, by weight. In yet another preferred embodiment, the aldehyde composition comprises greater than about 0.4 percent, more preferably, greater than about 0.6 percent tri-aldehyde, that is triformyl-substituted fatty acids or fatty acid esters, by weight. In another embodiment, the aldehyde composition comprises less than about 28 percent, preferably, less than about 23 percent tri-aldehyde, by weight.

In a preferred embodiment, the aldehyde composition is characterized by a di-aldehyde to tri-aldehyde (di-al/tri-al) weight ratio less than 4.5/1, preferably, less than about 4.0/1.

In a more preferred embodiment, the aldehyde composition comprises greater than about 3 percent saturates, even more preferably, greater than about 5 percent saturates, and most preferably, greater than about 10 percent saturates. In a more preferred embodiment, the aldehyde composition comprises less than about 30 percent saturates. For the purposes of this invention, the term “saturates” includes any fatty acid or fatty acid ester wherein each carbon atom in the fatty acid chain is covalently bonded to four other atoms (that is, no carbon-carbon double or triple bonds are present), with the added requirement that the saturates do not contain any formyl or hydroxy substituents, excepting those that might naturally occur in the seed oil.

In another more preferred embodiment, the aldehyde composition comprises greater than about 1 percent unsaturates. In another more preferred embodiment, the aldehyde composition comprises less than about 20 percent unsaturates. For the purposes of this invention, the term “unsaturates” refers to any fatty acid or fatty acid ester that contains at least one carbon-carbon double bond, with the added requirement that such compounds do not contain any formyl or hydroxymethyl substituents, excepting those that might naturally occur in the seed oil.

In yet another preferred embodiment, the aldehyde composition comprises less than about 10 weight percent impurities, for example heavies, as described hereinafter.

This invention also provides for a process of preparing the novel aldehyde composition described hereinabove, comprising contacting a mixture of unsaturated fatty acids and/or fatty acid esters with carbon monoxide and hydrogen in the presence of a Group VIII transition metal-organophosphine metal salt ligand complex catalyst, and optionally free organophosphine metal salt ligand, under process conditions sufficient to hydroformylate, typically, greater than about 79 weight percent, and preferably, greater than about 83 weight percent and less than about 99 weight percent, of unsaturated fatty acids or fatty acid esters to monoaldehyde products, so as to obtain a mixture of formyl-substituted fatty acids or fatty acid esters comprising in terms of formyl distribution from greater than about 30 to less than about 95 percent mono-aldehyde, from greater than about 0.4 to less than about 37 percent di-aldehyde, and from greater than about 0.1 to less than about 34 percent tri-aldehyde, by weight, based on the total weight of the aldehyde composition; the mixture also having a di-al/tri-al weight ratio less than 5/1 and an average functionality number ranging from greater than 0.96 to less than 1.26.

In a second aspect, this invention provides for a novel alcohol composition comprising a mixture of hydroxymethyl-substituted fatty acids and/or fatty acid esters comprising in terms of hydroxy distribution from greater than about 30 to less than about 90 percent monoalcohol, from greater than about 0.4 to less than about 34 percent dialcohol (diol), and from greater than about 0.1 to less than about 31 percent trialcohol (triol), by weight, based on the total weight of the composition. The alcohol composition of this invention is also characterized by a diol/triol weight ratio less than 5/1 and an average functionality number ranging from greater than 0.90 to less than 1.20.

For the purposes of this invention, the term “mono-alcohol” or “monol” refers to any fatty acid or fatty acid ester having one hydroxymethyl (—CH₂OH) substituent per molecule. The hydroxymethyl substituent may occur at any saturated carbon atom along the fatty acid chain, which itself may be fully saturated or may additionally contain one or more unsaturated C═C double bonds. The unsaturated C═C double bonds are those that were present in the seed oil, but which remained unconverted in the process (hydroformylation/hydrogenation) of producing the mono-alcohol. Likewise, the terms “dialcohol” and “trialcohol” refer to any fatty acid or fatty acid ester having two or three hydroxymethyl substituents, respectively, per molecule. The di- and tri-hydroxymethyl substituents may be distributed among the saturated carbon atoms along the fatty acid chain. Likewise, the fatty acid or fatty acid ester chain of the dialcohol or trialcohol may be fully saturated or additionally may contain one or more unsaturated C═C double bonds, although unsaturated trialcohol may be less likely to occur. It is further noted that the words “mono-alcohol,” “dialcohol,” and “trialcohol” each individually include single species thereof or mixtures of such species differentiated by fatty acid chains of different lengths. For example, the term “mono-alcohol” can refer to a single species of C₁₆ mono-alcohol or can refer to a mixture of C₁₆ and C₁₈ mono-alcohols.

As applied to the novel alcohol composition, the term “average functionality number” is defined as the average number of hydroxymethyl functionality per alcohol chain, as explained in more detail hereinafter.

In a preferred embodiment, the alcohol composition comprises greater than about 40 percent, more preferably, greater than about 50 percent mono-alcohol, that is, mono-hydroxymethyl-substituted fatty acid(s) or fatty acid ester(s), by weight, based on the total weight of the composition. In a preferred embodiment, the alcohol composition comprises less than about 88 percent, more preferably, less than about 86 percent mono-alcohol, by weight. In a preferred embodiment, the alcohol composition comprises greater than about 1 percent, and more preferably, greater than about 2 percent dialcohol, that is, dihydroxymethyl-substituted fatty acid(s) or fatty acid ester(s), by weight. In a preferred embodiment, the alcohol composition comprises less than about 29 percent, and more preferably, less than about 24 percent dialcohol, by weight. In a preferred embodiment, the alcohol composition comprises greater than about 0.4 percent, and more preferably, greater than about 0.6 percent trialcohol, that is, trihydroxymethyl-substituted fatty acid(s) or fatty acid ester(s), by weight. In a preferred embodiment, the alcohol composition comprises less than about 26 percent, and more preferably, less than about 20 percent trialcohol, by weight.

In a more preferred embodiment, the alcohol composition comprises greater than about 3 percent, even more preferably, greater than about 5 percent, and most preferably, greater than about 10 percent saturates, by weight. In a more preferred embodiment, the alcohol composition comprises less than about 35 percent, and most preferably, less than about 30 percent saturates, by weight. The term “saturates” is given the same meaning as set forth hereinabove, referring to any fatty acid or fatty acid ester wherein each carbon atom in the fatty acid chain is covalently bonded to four atoms (that is, no carbon-carbon double or triple bonds are present), with the added requirement that the saturates do not contain any formyl or hydroxymethyl substituents, excepting those that might naturally occur in the seed oil.

In another more preferred embodiment, the alcohol composition comprises less than about 10 percent unsaturates, by weight. The term “unsaturates” has the same meaning as set forth hereinabove with reference to any fatty acid or fatty acid ester that contains at least one carbon-carbon double bond, with the added requirement that such components do not contain any formyl or hydroxymethyl substituents, excepting those that might naturally occur in the seed oil.

In yet another preferred embodiment, the alcohol composition is characterized by a diol/triol weight ratio of less than about 4.5/1, preferably, less than about 4.0/1.

In yet another preferred embodiment, the alcohol composition comprises less than about 12 weight percent impurities, including lactols, lactones, saturated and unsaturated cyclic ethers, and heavies, as described hereinafter.

This invention also provides for a process of preparing the novel alcohol composition comprising (a) contacting a mixture comprising unsaturated fatty acids and/or fatty acid esters with carbon monoxide and hydrogen in the presence of a Group VIII transition metal-organophosphine metal salt ligand complex catalyst, and optionally, free organophosphine metal salt ligand, under conditions sufficient to hydroformylate typically greater than about 79 weight percent, and preferably greater than about 83 weight percent and less than about 99 weight percent, unsaturated fatty acids or fatty acid esters to monoformyl products, so as to obtain a hydroformylation reaction mixture comprising an aldehyde product of formyl-substituted fatty acids or fatty acid esters; (b) separating the aldehyde product from the hydroformylation reaction mixture; and thereafter (c) hydrogenating the aldehyde product with a source of hydrogen in the presence of a hydrogenation catalyst under process conditions sufficient to obtain the alcohol composition comprising in terms of hydroxy distribution from greater than about 30 to less than about 90 percent monoalcohol, from greater than about 0.4 to less than about 34 percent dialcohol, and from greater than about 0.1 to less than about 31 percent trialcohol, by weight, based on the total weight of the composition, the composition also having a diol/triol weight ratio less than 5/1 and an average functionality number ranging from greater than 0.90 to less than 1.20.

For the purposes of this invention, the term “average functionality number” (AFN) shall mean the average number of formyl or hydroxymethyl functionality per aldehyde or alcohol monomer composition, respectively. Each sample of aldehyde or alcohol composition may be expressed as comprising the following components:

A+B+C+D+E+F=1.0 (Eq. 1)

wherein
A=mole fraction of saturates;
B=mole fraction of mono-aldehyde or mono-alcohol;
C=mole fraction of di-aldehyde or diol;
D=mole fraction of tri-aldehyde or triol;
E=mole fraction of lactols, lactones, and cyclic ethers;
F=mole fraction of dimers and heavies.
   Based on the above composition, the average functionality number (AFN) can be calculated as follows:

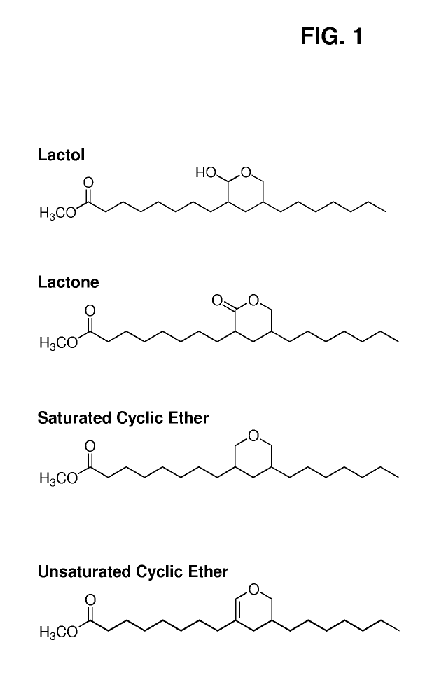

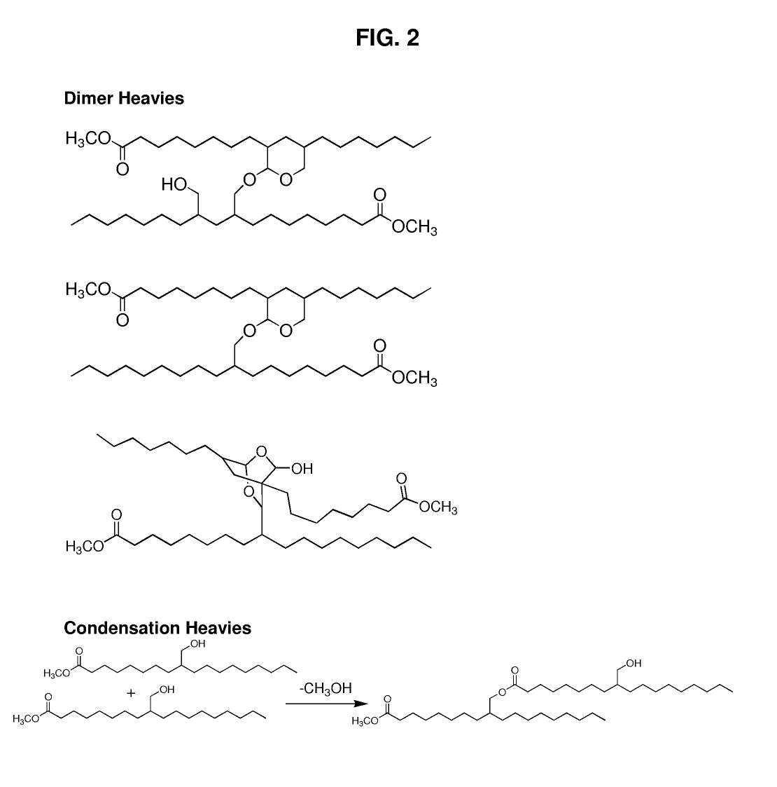

AFN=0A+1B+2C+3D+1E+2F (Eq. 2)

wherein each mole fraction is multiplied by the number of formyl or hydroxymethyl functionalities per fatty acid chain of that fraction. More explicitly, the number of formyl or hydroxymethyl functionalities per fraction is as follows: (A) 0 for unsaturates, (B) 1 for mono-als and mono-ols, (C) 2 for di-als and diols, (D) 3 for tri-als and triols. Fraction E comprising lactols, lactones, and cyclic ethers is taken to have a functionality of 1. Fraction F comprising dimers and heavies is taken to have a functionality of 2. (FIGS. 1 and 2 illustrate structures of possible components in Fractions E and/or F, such structures being based upon molecular weights obtained via mass spectroscopy analysis and expected chemical reactions of formyl and hydroxy-methyl-substituted components. Cyclic ethers are believed to be produced by dehydration of lactols during gas chromatographic analysis of the sample.) Since the saturates (A) contribute no functionality, the first term of Equation 2 is zero; and equation (2) is reduced to the following:

AFN=1B+2C+3D+1E+2F (Eq. 3)

Typically, the mole fractions of mono-, di-, and tri-substituted components (B, C, and D) can be based upon the molecular weight of the C₁₈ component of the seed oil. Typically, the C₁₆ and C₂₀ components occur in small quantities that may, in fact, balance each other out in the calculation of mole fraction. Such a guideline should not, however, be taken as a requirement of this invention. In seed oils, wherein a C₁₆, C₂₀, or other carbon chain other than C₁₈ occurs in significant quantity, it may be necessary to separate every component of the mono-, di-, and tri-substituted fractions and calculate their individual contributions to the mole fractions of B, C, and D.

For any given aldehyde composition that is converted via hydrogenation into the corresponding alcohol composition, a difference is typically found between the average functionality number of the aldehyde composition and the average functionality number of the alcohol composition. Such a difference is not necessarily expected, but may arise from differences in the analyses of weight percentages of corresponding mono-, di-, and tri-substituted components in the aldehyde composition versus the alcohol composition. These differences are derived from factors influencing the analysis of the composition of the sample; for example, an aldehyde component may have a different response factor and associated error factor in a gas chromatographic analysis, as compared with the corresponding alcohol component. Moreover, using current best technology available, the average functionality number of both the aldehyde and the alcohol compositions has an associated error of +/−0.04.

The average functionality number of the alcohol composition may also be determined empirically by means of American Standard Test Method 4274 for determining hydroxyl number. Generally, the empirical method correlates closely with the calculated method. Such an empirical method is not at the present time available for determining the average functionality number of the aldehyde composition.

When the average functionality number of the alcohol composition ranges between 0.90 and 1.20, and the diol/triol weight ratio is less than 5/1, then such alcohol compositions as may be derived, e.g., from canola oil, rapeseed oil, or a mixture of oils, can be suitably employed as monomers in the preparation of polyols for use in polyurethane flexible foams.

The fatty acid or fatty acid ester feedstock suitably employed in preparing the aldehyde and alcohol compositions of this invention is preferably derived from natural and genetically modified (GMO) plant and vegetable seed oils. Suitable non-limiting examples of such seed oils include canola and rapeseed, including genetically-modified variations thereof; as well as mixtures of various other oils falling within the compositional limitations of this invention, for example, mixtures of soy and linseed oils, mixtures of canola and linseed oils, and mixtures of peanut and linseed oils. Preferably, the fatty acid or fatty acid ester feedstock is derived from canola oil or mixtures of linseed oil with other seed oils.

Typically, each fatty acid component of the seed oil comprises a fatty acid chain of greater than about 5, preferably, greater than about 10, and more preferably, greater than about 12 carbon atoms. Typically, the fatty acid chain contains less than about 50, preferably, less than about 35, and more preferably, less than about 25 carbon atoms. The fatty acid chain may be straight or branched and substituted with one or more substituents, provided that the substituents do not materially interfere with the processes described herein and any desired downstream end-use. Non-limiting examples of suitable substituents include alkyl moieties, preferably C₁₋₁₀ alkyl moieties, for example methyl, ethyl, propyl, and butyl; cycloalkyl moieties, preferably, C₄₋₈ cycloalkyl; phenyl; benzyl; C₇₋₁₆ alkaryl and aralkyl moieties; hydroxy, ether, keto, and halide (preferably, chloro and bromo) substituents.

Seed oils comprise a mixture of both saturated and unsaturated fatty acids and/or fatty acid esters. For use in this invention, typically, the seed oil comprises greater than about 75 percent, preferably, greater than about 85 percent, and more preferably, greater than about 95 percent unsaturated fatty acids and/or fatty acid esters. Any distribution of mono-, di-, and tri-unsaturation in the seed oil may be suitably employed, provided that the aldehyde and alcohol compositions of this invention are obtainable therefrom. As a guideline, the seed oil typically comprises from greater than about 50 to less than about 90 percent mono-unsaturated fatty acids and/or fatty acid esters; from greater than about 1 to less than about 45 percent di-unsaturated fatty acids and/or fatty acid esters; and from greater than about 0.4 to less than about 45 percent tri-unsaturated fatty acids and/or fatty acid esters, by weight. Preferably, a mixture of fatty acid and/or fatty acid esters is employed wherein the weight ratio of di-unsaturates to tri-unsaturates is less than about 3:1. Typically, the weight ratio of di-unsaturates to tri-unsaturates is greater than about 0.1:1.

Non-limiting examples of suitable unsaturated fatty acids that may be found in the seed oil feedstock include 3-hexenoic (hydrosorbic), trans-2-heptenoic, 2-octenoic, 2-nonenoic, cis- and trans-4-decenoic, 9-decenoic (caproleic), 10-undecenoic (undecylenic), trans-3-dodecenoic (linderic), tridecenoic, cis-9-tetradeceonic (myristoleic), pentadecenoic, cis-9-hexadecenoic (cis-9-palmitoelic), trans-9-hexadecenoic (trans-9-palmitoleic), 9-heptadecenoic, cis-6-octadecenoic (petroselinic), trans-6-octadecenoic (petroselaidic), cis-9-octadecenoic (oleic), trans-9-octadecenoic (elaidic), cis-11-octadecenoic, trans-11-octadecenoic (vaccenic), cis-5-eicosenoic, cis-9-eicosenoic (godoleic), cis-11-docosenoic (cetoleic), cis-13-docosenoic (erucic), trans-13-docosenoic (brassidic), cis-15-tetracosenoic (selacholeic), cis-17-hexacosenoic (ximenic), and cis-21-triacontenoic (lumequeic) acids, as well as 2,4-hexadienoic (sorbic), cis-9-cis-12-octadecadienoic (linoleic), cis-9-cis-12-cis-15-octadecatrienoic (linolenic), eleostearic, 12-hydroxy-cis-9-octadecenoic (ricinoleic), cis-5-docosenoic, cis-5,13-docosadienoic, 12,13-epoxy-cis-9-octadecenoic (vemolic), and 14-hydroxy-cis-ii-eicosenoic acid (lesquerolic) acids. The most preferred unsaturated fatty acid is oleic acid.

In seed oils the alcohol segment of the fatty acid ester is glycerol, a trihydric alcohol. Generally, the fatty acid esters employed in preparing the aldehyde or alcohol compositions of this invention are obtained by transesterifying a seed oil with a lower alkanol. Transesterification produces the corresponding mixture of saturated and unsaturated fatty acid esters of the lower alkanol. Since glycerides can be difficult to process and separate, transesterification of the seed oil with a lower alkanol yields mixtures that are more suitable for chemical transformations and separation. Typically, the lower alcohol has from 1 to about 15 carbon atoms. The carbon atoms in the alcohol segment may be arranged in a straight-chain or a branched structure, and may be substituted with a variety of substituents, such as those previously disclosed hereinabove in connection with the fatty acid segment, provided that such substituents do not interfere with processing and downstream applications. Preferably, the alcohol is a straight-chain or a branched C₁₋₈ alkanol, more preferably, a C₁₋₄ alkanol. Even more preferably, the lower alkanol is selected from methanol, ethanol, and isopropanol. Most preferably, the lower alkanol is methanol.

Any known transesterification method can be suitably employed, provided that the ester products of the lower alkanol are achieved. The art adequately discloses transesterification (for example, methanolysis, ethanolysis) of seed oils; for example, refer to WO 2001/012581, DE 19908978, and BR 953081. Typically, in such processes, the lower alkanol is contacted with alkali metal, preferably sodium, at a temperature between about 30° C. and about 100° C. to prepare the corresponding metal alkoxide. Then, the seed oil is added to the alkoxide mixture, and the resulting reaction mixture is heated at a temperature between about 30° C. and about 100° C. until transesterification is effected. The crude transesterified composition may be separated from the reaction mixture by methods known in the art, including for example, phase separation, extraction, and/or distillation. The crude product may also be separated from co-products and/or decolorized using column chromatography, for example, with silica gel. Variations on the above procedure are documented in the art.

If a mixture of fatty acids, rather than fatty acid esters, is desirably employed as the feedstock for this invention, then the selected seed oil can be hydrolyzed to obtain the corresponding mixture of fatty acids. Methods for hydrolyzing seed oils to their constituent fatty acids are also well known in the art.

Although the description herein refers in the alternative to fatty acids or fatty acid esters, the description does not intend to exclude the possibility of using and obtaining mixtures of fatty acids and fatty acid esters. Preferably, on a practical level, the compositions comprise essentially acids or essentially esters; but as noted a mixture thereof is also conceivable.

In preparing the aldehyde composition of this invention, the mixture of fatty acids or fatty acid esters derived from the seed oil is subjected to hydroformylation. It is preferred to employ non-aqueous hydroformylation processes that employ the operational features taught in U.S. Pat. No. 4,731,486, U.S. Pat. No. 4,633,021 and WO 2004/0963744; the disclosures of said patents being incorporated herein by reference. Accordingly, another aspect of this invention comprises contacting the mixture of unsaturated fatty acids or fatty acid esters derived from the seed oil with carbon monoxide and hydrogen in a non-aqueous reaction medium in the presence of a solubilized Group VIII transition metal-organophosphine metal salt ligand complex catalyst, and optionally solubilized free organophosphine metal salt ligand, under conditions sufficient to prepare the aldehyde composition described herein. The term “non-aqueous reaction medium” means that the reaction medium is essentially free of water, which means that to the extent that water is present at all, it is not present in an amount sufficient to cause the hydroformylation reaction mixture to be considered as encompassing a separate aqueous or water phase or layer in addition to the organic phase. The term “free” organophosphine metal salt ligand means that the organophosphine metal salt ligand is not complexed, that is, not bound or tied to the Group VIII transition metal.

The Group VIII transition metals are selected from the group consisting of iron (Fe), cobalt (Co), nickel (Ni), ruthenium (Ru), rhodium (Rh), palladium (Pd), osmium (Os), iridium (Tr), and platinum (Pt), and mixtures thereof; with the preferred metals being rhodium, ruthenium, cobalt, and iridium; more preferably, rhodium and cobalt; and most preferably, rhodium. The oxidation state of the Group VIII metal may be any available oxidation state, either electronically neutral (zero) or electronically deficient (positive valence), that allows for bonding to the organophosphine ligand. Moreover, the oxidation state of the Group VIII transition metal, as well as the overall oxidation state of the complex or any complex precursor, may vary under the hydroformylation process conditions. The term “complex” as used herein shall be taken to mean a coordination compound formed by the union of one or more organophosphine ligands with the Group VIII transition metal. The number of available coordination sites on the Group VIII transition metal is well known in the art and may range typically from about 4 to about 6. Optionally, the Group VIII transition metal may be additionally bonded to carbon monoxide, hydrogen, or both carbon monoxide and hydrogen. In general, the Group VIII transition metal is employed in the hydroformylation process in a concentration range of from about 10 parts per million (ppm) to about 1000 ppm, by weight, calculated as free metal. In rhodium catalyzed hydroformylation processes, it is generally preferred to employ from about 10 to about 800 ppm of rhodium calculated as free metal.

The organophosphine metal salt ligand preferably employed in the hydroformylation process of this invention comprises a monosulfonated tertiary phosphine metal salt, preferably, represented by formula I hereinafter:

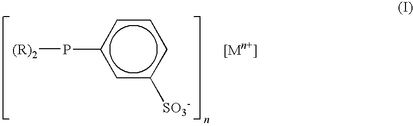

wherein each R group individually represents a radical containing from 1 to about 30 carbon atoms selected from the classes consisting of alkyl, aryl alkaryl, aralkyl, and cycloalkyl radicals; wherein M represents a metal cation selected from the group consisting of alkali and alkaline earth metals; and wherein n has a value of 1 or 2 corresponding to the valence of the particular metal cation M. Non-limiting examples of monosulfonated tertiary phosphine metal salt ligands of the aforementioned structure are illustrated in the art, for example, in U.S. Pat. No. 4,731,486, incorporated herein by reference. More preferred ligands are selected from monosulfonated metal salt derivatives of triphenylphosphine, diphenylcyclohexylphosphine, phenyldicyclohexyphosphine, tricyclohexylphosphine, diphenylisopropylphosphine, phenyldiisopropylphosphine, diphenyl-t-buylphosphine, phenyldi-t-butylphosphine, and the like. A most preferred ligand is selected from the monosulfonated metal salt derivatives of dicyclohexylphenylphosphine.

The hydroformylation process of this invention may be conducted in an excess amount of free ligand, for example, at least one mole of free monosulfonated tertiary organophosphine metal salt ligand per mole of Group VIII transition metal present in the reaction medium. In general, amounts of free ligand from about 2 to about 300, and preferably, from about 5 to about 200 moles per mole of Group VIII transition metal present in the reaction medium should be suitable for most purposes, particularly with regard to rhodium catalyzed processes. If desired, make-up organophosphine ligand can be supplied to the reaction medium or the hydroformylation process at any time and in any suitable manner, so as to maintain preferred concentrations of free ligand in the reaction medium.

The monosulfonated tertiary phosphine metal salt ligands mentioned hereinabove are generally water soluble, and not soluble or very poorly soluble in most olefins and/or aldehydes, and particularly, not soluble or very poorly soluble in the unsaturated fatty acids or fatty acid esters and formyl derivatives thereof under consideration in this invention. It is known, however, that by use of certain organic solubilizing agents, the monosulfonated tertiary phosphine metal salt ligand and Group VIII complexes thereof can be rendered organically soluble and thus employable in non-aqueous hydroformylation reaction media. Organic solubilizing agents used for the aforementioned purpose are disclosed in the prior art, for example, in U.S. Pat. No. 5,180,854 and U.S. Pat. No. 4,731,486, incorporated herein by reference. U.S. Pat. No. 5,180,854 discloses as organic solubilizing agents amides, glycols, sulfoxides, sulfones, and mixtures thereof. N-methyl-2-pyrrolidinone (NMP) is one preferred organic solubilizing agent. As disclosed in U.S. Pat. No. 4,731,486, other suitable polar solvents or solubilizing agents include alkylene oxide oligomers having an average molecular weight greater than about 150 up to about 10,000, and higher; organic nonionic surfactant mono-ols having an average molecular weight of at least about 300; and alcohol alkoxylates containing both water-soluble (polar) and oil-soluble (non-polar) groups readily available under the trademark TERGITOL.

The reaction conditions for affecting the non-aqueous hydroformylation process can vary widely over conventional ranges; however, the conversion of unsaturated fatty acid(s) and/or fatty acid ester(s), as discussed hereinbelow, constitutes an important factor in providing for the compositions described herein. A reaction temperature typically greater than about 45° C., and preferably, greater than about 60° C. can be suitably employed. The hydroformylation process, however, typically operates at a temperature less than about 200° C., and preferably, less than about 130° C. Such a process generally operates at a total pressure greater than about 1 psia (6.9 kPa), preferably, greater than about 50 psia (345 kPa). Typically, the process operates at a total pressure less than about 10,000 psia (69 MPa), preferably, less than about 1,500 psia (10 MPa), and more preferably, less than about 500 psia (3.5 MPa). The minimum total pressure of the reactants is not particularly critical and depends predominately on the amount and nature of the reactants employed to obtain a desired rate of reaction. More specifically, the carbon monoxide partial pressure is preferably greater than about 1 psia (6.9 kPa), and more preferably, greater than about 25 psia (172 kPa). The carbon monoxide partial pressure is preferably less than about 250 psia (1,724 kPa), and more preferably, less than about 200 psia (1,379 kPa). The hydrogen partial pressure preferably is greater than about 10 psia (69 kPa), more preferably, greater than about 25 psia (172 kPa). The hydrogen partial pressure is preferably less than about 250 psia (1,724 kPa), and more preferably, less than about 200 psia (1,379 kPa). In general, the molar ratio of gaseous hydrogen to carbon monoxide (H₂:CO) can range from about 1:10 to about 10:1. The reaction medium residence time typically ranges from greater than about 1 hour to less than about 40 hours per reactor. The hydroformylation process can be operated as a batch process, or preferably, conducted as a continuous process with recycle of the complex catalyst and optional free ligand. A preferred reactor comprises from 1 to about 5 continuous stirred tank reactors connected in series. Each stirred tank reactor may contain one or multiple stages, as desired. Other engineering variations are known and described in the art.

As mentioned hereinabove, the conversion of unsaturated fatty acid(s) and/or fatty acid ester(s) in the hydroformylation process provides a tool for obtaining the compositions of this invention. Mixtures of unsaturated fatty acids and/or unsaturated fatty acid esters can be analyzed by gas phase chromatographic (GC) methods known to those of skill in the art. The conversion of unsaturated fatty acid(s) and/or fatty acid ester(s) in organic processes, such as hydroformylation, can be tracked by such GC methods. Specifically, one or more GC peaks representative of the unsaturated fatty acids or fatty acid esters (that is, compounds with C═C double bonds and no formyl substituents) are typically found to decrease in peak height and peak area as the hydroformylation progresses. The extent of this peak loss can be correlated with the conversion of unsaturated fatty acids or fatty acid esters first to monoformyl-substituted products. Some monoformyl products containing additional unsaturation will be involved in a secondary reaction to diformyl products; and a portion of the diformyl products containing additional unsaturation will be involved in a tertiary reaction to triformyl products. For the purposes of this invention, these secondary and tertiary reactions to diformyl and triformyl products are not considered in the calculation of conversion. Rather, consideration is given only to the conversion of the first unsaturated bond per molecule of unsaturated fatty acid or fatty acid ester to monoformyl product. Under the process conditions described hereinbefore, the hydroformylation process is conducted to a conversion of greater than about 79 weight percent, preferably, greater than about 83 weight percent unsaturated fatty acids or fatty acid esters, based on the conversion of one unsaturated bond per molecule. Preferably, the conversion is less than about 99 weight percent, and more preferably, less than about 97 weight percent unsaturated fatty acids or fatty acid esters, based on the conversion of one unsaturated bond per molecule. Note that by the instant definition the conversion is not equivalent to the percent conversion of all unsaturated bonds.

When the hydroformylation process is conducted as described hereinabove, then an aldehyde composition is obtained that comprises a mixture of formyl-substituted fatty acids or fatty acid esters having the following composition by weight: from greater than about 30 to less than about 95 percent monoaldehyde, from greater than about 0.4 to less than about 37 percent di-aldehyde, and from greater than about 0.1 to less than about 34 percent tri-aldehyde-substituted fatty acids or fatty acid esters; preferably, from greater than about 3 to less than about 30 percent saturates; preferably, from greater than about 1 to less than about 20 percent unsaturates; and preferably, less than about 10 percent impurities, by weight. In addition, the aldehyde composition has a di-al/tri-al weight ratio typically less than 5/1, preferably, less than 4.5/1, and more preferably, less than 4.0/1. Further, the aldehyde composition has an average number, more particularly, an average formyl number, ranging from greater than 0.96 to less than 1.26.

The formyl-substituted fatty acids or fatty acid esters may contain impurities including heavies. Typically, the total concentration of impurities is greater than about 0.01 weight percent, based on the total weight of the aldehyde composition. Preferably, the total concentration of impurities is less than about 10, preferably, less than about 5, and more preferably, less than about 2 weight percent, based on the total weight of the aldehyde composition. Generally, it is desirable to maintain a low level of these impurities, because their presence may impact the properties of manufactured downstream end-products.

The aldehyde composition can be separated from the hydroformylation reaction medium, the Group VIII transition metal-organophosphine metal salt ligand complex catalyst, and free organophosphine metal salt ligand by methods known in the art. Extraction is a preferred method of separation. A suitable extraction method is described in U.S. Pat. No. 5,180,854, incorporated herein by reference. The extraction method disclosed therein comprises mixing the non-aqueous reaction mixture with from about 2 to about 60 percent by weight of added water and from 0 to about 60 percent by weight of a non-polar hydrocarbon, and then by phase separation forming a non-polar phase comprising the aldehyde composition and the non-polar hydrocarbon compound, if any, and a liquid polar phase comprising water, the Group VIII transition metal-organophosphine metal salt ligand complex catalyst, optionally free organophosphine metal salt ligand, and any organic solubilizing agent. Typically, the non-polar hydrocarbon comprises a saturated straight chain alkane containing from about 6 to about 30 carbon atoms, such as, hexane. The aldehyde composition may be processed directly in the non-polar hydrocarbon, or of desired, may be separated by conventional methods from the non-polar hydrocarbon. The hydroformylation complex catalyst and organophosphine ligand are typically extracted from the liquid polar phase and recycled back to the hydroformylation reactor. As a result of the above-described hydroformylation and separation procedures, the aldehyde composition may additionally comprise small quantities of water, hydroformylation solvent, solubilizing agent, and/or extraction solvent.

The conversion of aldehydes to alcohols is known in the art, and such conventional methods can be applied to convert the aldehyde composition of this invention to the alcohol composition of this invention. Typically, the aldehyde composition comprising the mixture of formyl-substituted fatty acids or fatty acid esters is contacted with a source of hydrogen in the presence of a hydrogenation catalyst under hydrogenation process conditions sufficient to prepare the alcohol composition comprising a mixture of hydroxymethyl-substituted fatty acids or fatty acid esters. The source of hydrogen may be pure hydrogen or hydrogen diluted with a non-reactive gas, such as nitrogen, helium, argon, a saturated hydrocarbon, or the like. The hydrogenation catalyst may be any such catalyst capable of converting the aldehyde composition to the alcohol composition. Preferably, the hydrogenation catalyst comprises a metal selected from Group VIII, Group IB, and Group IIB of the Periodic Table, and mixtures thereof; more preferably, a metal selected from palladium, platinum, rhodium, nickel, copper, and zinc, and mixtures thereof. The metal may be supplied as Raney metal or as metal supported on a suitable catalyst support, such as carbon or silica. An even more preferred hydrogenation catalyst is Raney nickel or supported nickel. The hydrogenation may be conducted neat or in a solution of a suitable hydrocarbon solvent. The temperature for such hydrogenations is generally greater than about 50° C., and preferably, greater than about 80° C. The temperature for such hydrogenations is typically less than about 250° C., and preferably, less than about 175° C. The hydrogen pressure is generally greater than about 50 psig (345 kPa). The hydrogen pressure is generally less than about 1,000 psig (6,895 kPa), and preferably, less than about 600 psig (4,137 kPa).

The alcohol composition of this invention can also be obtained as a mixture by mixing together two or more different alcohol compositions obtained from separate hydrogenation processes. The mixture, for example, can be prepared by mixing various alcohol compositions falling within the scope of this invention. Alternatively, the mixture can be prepared by mixing two more alcohol compositions lying outside the scope of this invention. For example, an alcohol composition of hydroxymethyl-substituted fatty acids or fatty acid esters having an average functionality less than 0.90 can be mixed with an alcohol composition of hydroxymethyl-substituted fatty acids or fatty acid esters having an average functionality greater than 1.20 to arrive at an alcohol composition having an average functionality falling within the claimed range, namely, greater than 0.90 and less than 1.20. Likewise, the alcohol composition can be prepared by mixing an alcohol composition falling within the scope of the claims with an alcohol composition falling outside the scope of the claims to arrive again at a composition falling within the scope of the claims.

The hydrogenation conducted as described hereinabove produces the alcohol composition comprising a mixture of hydroxymethyl-substituted fatty acids or fatty acid esters comprising in terms of hydroxy distribution from greater than about 30 to less than about 90 percent monoalcohol, from greater than about 0.4 to less than about 34 percent diol, and from greater than about 0.1 to less than about 31 percent triol; preferably, from greater than about 3 to less than about 35 percent saturates; and preferably, less than about 10 percent unsaturates. The alcohol composition is further characterized as comprising a diol to triol weight ratio of less than 5/1 and an average functionality number (i.e., average hydroxymethyl number) ranging from greater than 0.90 to less than 1.20.

The alcohol composition may contain impurities, such as lactols, lactones, saturated and unsaturated cyclic ethers, and heavies, for example, having the structures shown in FIGS. 1 and 2 for a fatty acid of carbon chain length 18. Analogous species may be present based on fatty acids or fatty acid esters having different substitution or having chain lengths different from 18. Typically, the concentration of lactols and/or lactones is greater than about 0.01 weight percent, based on the total weight of the alcohol composition. Typically, the concentration of lactols and/or lactones is less than about 20, and preferably, less than about 10 weight percent, based on the total weight of the alcohol composition. Typically, the concentration of unsaturated and/or saturated cyclic ethers is greater than about 0.01 weight percent, based on the total weight of the alcohol composition. Typically, the concentration of unsaturated and/or saturated cyclic ethers is less than about 10 weight percent, based on the total weight of the alcohol composition. Typically, the concentration of heavies is greater than about 0.01 weight percent, based on the total weight of the alcohol composition. Typically, the concentration of heavies is less than about 10 weight percent, based on the total weight of the alcohol composition. Typically, the total concentration of impurities is greater than about 0.01 weight percent, based on the total weight of the alcohol composition. Preferably, the total concentration of impurities is less than about 10, preferably, less than about 5, and more preferably, less than about 2 weight percent, based on the total weight of the alcohol composition. Generally, it is desirable to maintain a low level of these impurities, because their presence may impact the properties of manufactured downstream end-products.

The resulting alcohol composition, which comprises a mixture of hydroxymethyl-substituted fatty acids and/or fatty acid esters, can be reacted as a monomer with an initiator compound using reaction techniques known in the art to form an oligomeric polyol that is useful for making polyurethanes, and flexible polyurethane foam in particular.

The initiator, which contains two or more hydroxyl, primary amine, or secondary amine groups, can be a polyol, an alkanol amine, or a polyamine. Initiators of particular interest are polyols. Polyether polyol initiators are useful, including polymers of ethylene oxide and/or propylene oxide having from 2-8, especially 2-4 hydroxyl groups per molecule and a molecular weight of about 90 to 6000, especially from about 200 to 3000.

The hydroxymethyl-containing polyester polyol so produced generally contains some unreacted initiator compound, and may contain unreacted hydroxymethylated fatty acids or fatty acid esters. Initiator compounds often react only monofunctionally or difunctionally with the fatty acids or esters, and the resulting polyester polyol often contains free hydroxyl or amino groups bonded directly to the residue of the initiator compound.

The resulting polyol may be alkoxylated, if desired, to introduce polyether chains onto one or more of the hydroxymethyl groups. The resulting polyol may also be aminated through reaction with ammonia or a primary amine, followed by hydrogenation, to replace the hydroxyl groups with primary or secondary amine groups. Primary or secondary amine groups can also be introduced by capping the polyester polyol with a diisocyanate, and then converting the terminal isocyanate groups so introduced to amino groups through reaction with water.

The polyol of the invention may be combined with one or more additional high equivalent weight polyols for use in making a polyurethane foam. Suitable such additional high equivalent weight polyols include polyether polyols and polyester polyols. Polyether polyols include, for example, polymers of propylene oxide, ethylene oxide, 1,2-butylene oxide, tetramethylene oxide, block and/or random copolymers thereof, and the like. Of particular interest are poly(propylene oxide) homopolymers, random copolymers of propylene oxide and ethylene oxide in which the poly(ethylene oxide) content is, for example, from about 1 to about 30 percent by weight, ethylene oxide-capped poly(propylene oxide) polymers and ethylene oxide-capped random copolymers of propylene oxide and ethylene oxide. For slabstock foam applications, such polyethers preferably contain 2 to 4, especially 2 to 3, mainly secondary hydroxyl groups per molecule and have an equivalent weight per hydroxyl group of from about 400 to about 3000, especially from about 800 to about 1750. For high resiliency slabstock and molded foam applications, such polyethers preferably contain 2 to 4, especially 2 to 3, mainly primary hydroxyl groups per molecule and have an equivalent weight per hydroxyl group of from about 1000 to about 3000, especially from about 1200 to about 2000. The polyether polyols may contain low terminal unsaturation (for example, less than 0.02 meq/g or less than 0.01 meq/g), such as those made using so-called double metal cyanide (DMC) catalysts, as described for example in U.S. Pat. Nos. 3,278,457, 3,278,458, 3,278,459, 3,404,109, 3,427,256, 3,427,334, 3,427,335, 5,470,813 and 5,627,120. Polyester polyols typically contain about 2 hydroxyl groups per molecule and have an equivalent weight per hydroxyl group of about 400 to about 1500. Polymer polyols of various sorts may be used as well. Polymer polyols include dispersions of polymer particles, such as polyurea, polyurethane-urea, polystyrene, polyacrylonitrile and polystyrene-co-acrylonitrile polymer particles in a polyol, typically a polyether polyol. Suitable polymer polyols are described in U.S. Pat. Nos. 4,581,418 and 4,574,137.

When additional high equivalent weight polyols are used, the polyol of the invention may constitute at least 10, at least 25, at least 35, at least 50, or at least 65 percent of the total weight of all high equivalent weight polyols. The polyol of the invention may constitute 75 percent or more, 85 percent or more, 90 percent or more, 95 percent or more, or even 100 percent of the total weight of all high equivalent weight polyols. For example, the polyol of the invention may constitute from about 20 to 65 percent, from 35 to 65 percent, from 65 to 100 percent, or from 80 to 100 percent of the total weight of high equivalent weight polyol(s).

The polyol component may contain one or more crosslinkers in addition to the high equivalent weight polyols described above. However, in many cases it is preferred to use reduced quantities of crosslinkers as compared with conventional polyether polyol-based foam formulations. If used, suitable amounts of crosslinkers are from about 0.1 to about 1 part by weight, especially from about 0.25 to about 0.5 part by weight, per 100 parts by weight high equivalent weight polyols.

For purposes of this invention “crosslinkers” are materials having three or more isocyanate-reactive groups per molecule and an equivalent weight per isocyanate-reactive group of less than about 400. Crosslinkers preferably contain from 3 to 8, especially from 3 to 4 hydroxyl, primary amine or secondary amine groups per molecule and have an equivalent weight of from about 30 to about 200, especially from about 50 to about 125. Examples of suitable crosslinkers include diethanol amine, monoethanol amine, triethanol amine, mono- di- or tri(isopropanol) amine, glycerine, trimethylol propane, pentaerythritol, and the like.

The polyol component used to make polyurethane foam may also contain one or more chain extenders, which for the purposes of this invention means a material having two isocyanate-reactive groups per molecule and an equivalent weight per isocyanate-reactive group of less than about 400, especially from about 31 to 125. The isocyanate-reactive groups are preferably hydroxyl, primary aliphatic or aromatic amine or secondary aliphatic or aromatic amine groups. Representative chain extenders include amines, ethylene glycol, diethylene glycol, 1,2-propylene glycol, dipropylene glycol, tripropylene glycol, ethylene diamine, phenylene diamine, bis(3-chloro-4-aminophenyl)methane, and 2,4-diamino-3,5-diethyl toluene. If used, chain extenders are typically present in an amount from about 1 to about 50, especially about 3 to about 25 parts by weight per 100 parts by weight high equivalent weight polyol. Chain extenders are typically omitted from slabstock and high resiliency slabstock foam formulations.

The organic polyisocyanate may be a polymeric polyisocyanate, aromatic isocyanate, cycloaliphatic isocyanate, or aliphatic isocyanate. Exemplary polyisocyanates include m-phenylene diisocyanate, tolylene-2-4-diisocyanate, tolylene-2-6-diisocyanate, hexamethylene-1,6-diisocyanate, tetramethylene-1,4-diisocyanate, cyclohexane-1,4-diisocyanate, hexahydrotolylene diisocyanate, naphthylene-1,5-diisocyanate, methoxyphenyl-2,4-diisocyanate, diphenylmethane-4,4′-diisocyanate, 4,4′-biphenylene diisocyanate, 3,3′-dimethoxy-4,4′-biphenyl diisocyanate, 3,3′-dimethyl-4-4′-biphenyl diisocyanate, 3,3′-dimethyldiphenyl methane-4,4′-diisocyanate, 4,4′,4″-triphenyl methane triisocyanate, a polymethylene polyphenylisocyanate (PMDI), tolylene-2,4,6-triisocyanate and 4,4′-dimethyldiphenylmethane-2,2′,5,5′-tetraisocyanate. Preferably the polyisocyanate is diphenylmethane-4,4′-diisocyanate, diphenylmethane-2,4′-diisocyanate, PMDI, tolylene-2-4-diisocyanate, tolylene-2-6-diisocyanate or mixtures thereof. Diphenylmethane-4,4′-diisocyanate, diphenylmethane-2,4′-diisocyanate and mixtures thereof are generically referred to as MDI, and all can be used. Tolylene-2-4-diisocyanate, tolylene-2-6-diisocyanate and mixtures thereof are generically referred to as TDI, and all can be used.

The amount of polyisocyanate used in making polyurethane is commonly expressed in terms of isocyanate index, i.e. 100 times the ratio of NCO groups to isocyanate-reactive groups in the reaction mixture (including those provided by water if used as a blowing agent). In the production of conventional slabstock foam, the isocyanate index typically ranges from about 96 to about 140, especially from about 105 to about 115. In molded and high resiliency slabstock foam, the isocyanate index typically ranges from about 50 to about 150, especially from about 85 to about 110.

The reaction of the polyisocyanate and the polyol component is conducted in the presence of a blowing agent. Suitable blowing agents include physical blowing agents such as various low-boiling chlorofluorocarbons, fluorocarbons, hydrocarbons and the like. Fluorocarbons and hydrocarbons having low or zero global warming and ozone-depletion potentials are preferred among the physical blowing agents. Chemical blowing agents that decompose or react under the conditions of the polyurethane-forming reaction are also useful. By far the most preferred chemical blowing agent is water, which reacts with isocyanate groups to liberate carbon dioxide and form urea linkages. Water is preferably used as the sole blowing agent, in which case about 1 to about 7, especially about 2.5 to about 5 parts, by weight, water are typically used per 100 parts, by weight, high equivalent weight polyol. Water may also be used in combination with a physical blowing agent, particularly a fluorocarbon or hydrocarbon blowing agent. In addition, a gas such as carbon dioxide, air, nitrogen or argon may be used as the blowing agent in a frothing process.

A surfactant is also used in the foam formulation. A wide variety of silicone surfactants as are commonly used in making polyurethane foams can be used in making the foams of this invention. Examples of such silicone surfactants are commercially available under the tradenames Tegostab™ (Th. Goldschmidt and Co.), Niax™ (GE OSi Silicones) and Dabco™ (Air Products and Chemicals). The amount of surfactant used will vary somewhat according to the particular application and surfactant that is used, but in general will be between 0.1 and 6 parts by weight per 100 parts by weight high equivalent weight polyol.

The foam formulation will generally include a catalyst. The selection of a particular catalyst package varies somewhat with the other ingredients in the foam formulation. The catalyst may catalyze the polyol-isocyanate (gelling) reaction or the water-isocyanate (blowing) reaction (when water is used as the blowing agent), or both. In making water-blown foams, it is typical to use a mixture of at least one catalyst that favors the blowing reaction and at least one other that favors the gelling reaction.

A wide variety of materials are known to catalyze polyurethane forming reactions, including tertiary amines, tertiary phosphines, various metal chelates, acid metal salts, strong bases, various metal alcoholates and phenolates and metal salts of organic acids. Catalysts of most importance are tertiary amine catalysts and organotin catalysts. Examples of tertiary amine catalysts include: trimethylamine, triethylamine, N-methylmorpholine, N-ethylmorpholine, N,N-dimethylbenzylamine, N,N-dimethylethanolamine, N,N,N′,N′-tetramethyl-1,4-butanediamine, N,N-dimethylpiperazine, 1,4-diazobicyclo-2,2,2-octane, bis(dimethylaminoethyl)ether, triethylenediamine and dimethylalkylamines where the alkyl group contains from 4 to 18 carbon atoms. Mixtures of these tertiary amine catalysts are often used. Examples of suitably commercially available surfactants include Niax™ A1 (bis(dimethylaminoethyl)ether in propylene glycol available from GE OSi Silicones), Niax™ B9 (N,N-dimethylpiperazine and N—N-dimethylhexadecylamine in a polyalkylene oxide polyol, available from GE OSi Silicones), Dabco™ 8264 (a mixture of bis(dimethylaminoethyl)ether, triethylenediamine and dimethylhydroxyethyl amine in dipropylene glycol, available from Air Products and Chemicals), and Dabco™ 33LV (triethylene diamine in dipropylene glycol, available from Air Products and Chemicals), Niax™ A-400 (a proprietary tertiary amine/carboxylic salt and bis(2-dimethylaminoethy)ether in water and a proprietary hydroxyl compound, available from GE OSi Silicones); Niax™ A-300 (a proprietary tertiary amine/carboxylic salt and triethylenediamine in water, available from GE OSi Specialties Co.); Polycat™ 58 (a proprietary amine catalyst available from Air Products and Chemicals), Polycat™ 5 (pentamethyl diethylene triamine, available from Air Products and Chemicals) and Polycat™ 8 (N,N-dimethyl cyclohexylamine, available from Air Products and Chemicals).

Examples of organotin catalysts are stannic chloride, stannous chloride, stannous octoate, stannous oleate, dimethyltin dilaurate, dibutyltin dilaurate, other organotin compounds of the formula SnRₙ(OR)₄₋ₙ, wherein R is alkyl or aryl and n is 0-2, and the like. Organotin catalysts are generally used in conjunction with one or more tertiary amine catalysts, if used at all. Organotin catalysts tend to be strong gelling catalysts, so they are less preferred than the tertiary amine catalysts and if used, are preferably used in small amounts, especially in high resiliency foam formulations. Commercially available organotin catalysts of interest include Dabco™ T-9 and T-95 catalysts (both stannous octoate compositions available from Air Products and Chemicals).

Catalysts are typically used in small amounts, for example, each catalyst being employed from about 0.0015 to about 5% by weight of the high equivalent weight polyol.

In addition to the foregoing components, the foam formulation may contain various other optional ingredients such as cell openers; fillers such as calcium carbonate; pigments and/or colorants such as titanium dioxide, iron oxide, chromium oxide, azo/diazo dyes, phthalocyanines, dioxazines and carbon black; reinforcing agents such as fiber glass, carbon fibers, flaked glass, mica, talc and the like; biocides; preservatives; antioxidants; flame retardants; and the like.

In general, the polyurethane foam is prepared by mixing the polyisocyanate and polyol composition in the presence of the blowing agent, surfactant, catalyst(s) and other optional ingredients as desired, under conditions such that the polyisocyanate and polyol composition react to form a polyurethane and/or polyurea polymer while the blowing agent generates a gas that expands the reacting mixture. The foam may be formed by the so-called prepolymer method (as described in U.S. Pat. No. 4,390,645, for example), in which a stoichiometric excess of the polyisocyanate is first reacted with the high equivalent weight polyol(s) to form a prepolymer, which is in a second step reacted with a chain extender and/or water to form the desired foam. Frothing methods (as described in U.S. Pat. Nos. 3,755,212; 3,849,156 and 3,821,130, for example), are also suitable. So-called one-shot methods (such as described in U.S. Pat. No. 2,866,744) are preferred. In such one-shot methods, the polyisocyanate and all polyisocyanate-reactive components are simultaneously brought together and caused to react. Three widely used one-shot methods which are suitable for use in this invention include slabstock foam processes, high resiliency slabstock foam processes, and molded foam methods.

Slabstock foam is conveniently prepared by mixing the foam ingredients and dispensing them into a trough or other region where the reaction mixture reacts, rises freely against the atmosphere (sometimes under a film or other flexible covering) and cures. In common commercial scale slabstock foam production, the foam ingredients (or various mixtures thereof) are pumped independently to a mixing head where they are mixed and dispensed onto a conveyor that is lined with paper or plastic. Foaming and curing occurs on the conveyor to form a foam bun. The resulting foams are typically from about 1 to about 5 pounds per cubic foot (pcf or lb/cu ft) (16-80 kg/m³) in density, especially from about 1.2 to about 2.0 pcf (19.2-32 kg/m³).

A preferred slabstock foam formulation according to the invention uses water as the primary or more preferably sole blowing agent, and produces a foam having a density of about 1.2 to about 2.0 pcf (19.2-32 kg/m³), especially about 1.2 to about 1.8 pcf (19.2-28.8 kg/m³). To obtain such densities, about 3 to about 6, preferably about 4 to about 5 parts by weight water are used per 100 parts by weight high equivalent weight polyol.

High resiliency slabstock (HR slabstock) foam is made in methods similar to those used to make conventional slabstock foam. HR slabstock foams are characterized in exhibiting a Bashore rebound score of 55% or higher, per ASTM 3574.03. These foams tend to be prepared using somewhat higher catalyst levels, compared to conventional slabstock foams, to reduce energy requirements to cure the foam. HR slabstock foam formulations blown only with water tend to use lower levels of water than do conventional slabstock formulations and thus produce slightly higher density foams. Water levels tend to be from about 2 to about 3.5, especially from about 2.5 to about 3 parts per 100 parts high equivalent weight polyols. Foam densities are typically from about 2 pounds per cubic foot (pcf) to about 5 pcf (32-80 kg/m³), especially from about 2.1 to about 3 pcf (33.6-48 kg/m³).

Molded foam can be made according to the invention by transferring the reactants (polyol composition including the polyol of the invention, polyisocyanate, blowing agent, and surfactant) to a closed mold where the foaming reaction takes place to produce a shaped foam. Either a so-called “cold-molding” process, in which the mold is not preheated significantly above ambient temperatures, or a “hot-molding” process, in which the mold is heated to drive the cure, can be used. Cold-molding processes are preferred to produce high resilience molded foam. Densities for molded foams tend to be in the range of 2.0 to about 5.0 pounds per cubic foot (32-80 kg/m³).

The polyols of the invention are also useful in making foam via a mechanical frothing process. In such processes, air, nitrogen or other gas is whipped into a reacting mixture containing the high equivalent weight polyol(s), a polyisocyanate, and optionally catalysts, surfactants as described before, crosslinkers, chain extenders and other components. The frothed reaction mixture is then typically applied to a substrate where it is permitted to cure to form an adherent cellular layer. A frothing application of particular importance is the formation of carpet with an attached polyurethane cushion. Such carpet-backing processes are described, for example, in U.S. Pat. Nos. 6,372,810 and 5,908,701.

The foam of the invention is useful as furniture cushioning, automotive seating, automotive dashboards, packaging applications, other cushioning and energy management applications, carpet backing, gasketing, and other applications for which conventional polyurethane foams are used. Foams having Air Flows greater than 1.0 ft³/min are preferred, greater than 1.6 ft³/min more preferred, and greater than 2.0 ft³/min most preferred.

The following examples are presented hereinbelow to illustrate the inventions described herein. The examples should not be construed to limit the inventions in any manner. Based on the description provided herein, variations and modifications of the examples will be apparent to those of skill in the art.

### General Method of Analyzing Aldehyde Composition

Samples are analyzed after addition of an internal standard (diglyme). Analysis is made by gas chromatography (GC) using a HP 6890 gas chromatograph with a DB-5 capillary column. A flame ionization detector (FID) is used, and calibration is made by the internal standard method. Response factors for the following components are obtained by direct calibration: methyl palmitate, methyl stearate, methyl oleate, methyl linoleate, and methyl formylstearate. Response factors for the remainder of the target components are obtained by analogy. Conversion, calculated as percent conversion, is determined by the disappearance of the sum of the methyl oleate, methyl linoleate, and methyl linolenate peaks.

### General Method of Analyzing Alcohol Composition

The alcohol composition is analyzed after dilution (dioxane) and addition of an internal standard (diglyme). Analysis is by GC using a HP 5890 gas chromatograph with a DB-5 capillary column. Detection is by FID, and calibration is made by the internal standard method. Response factors for the following components are obtained by direct calibration: methyl palmitate, methyl stearate, methyl formylstearate, and methyl hydroxymethylstearate. Response factors for the remainder of the target components are obtained by analogy. Conversion, calculated as percent conversion, is determined by the disappearance of the methyl formylstearate peak.

### General Method of Analyzing for Dimers and Heavies Impurities in Aldehyde and Alcohol Compositions

Samples are analyzed after dilution in dioxane. Analysis is by GC using a HP 6890 gas chromatograph and a ZB-1 capillary column run at 100-350° C. Detection is by FID; and the analysis uses a “Normalized Area Percent” method after splitting the chromatogram into two regions: a products region and a heavies region.

### General Method of Analyzing Polyurethanes

Properties of the polyols and polymers are measured according to ASTM D-3574-03.

### Example 1

A catalyst solution is prepared by dissolving dicarbonylacetylacetonato-rhodium (I) (16.0 g) and dicyclohexyl-(3-sulfonoylphenyl)phosphine mono-sodium salt (70.0 g) in N-methyl-2-pyrrolidinone (NMP) (930 g) under a nitrogen atmosphere. The resulting mixture is then transferred to a nitrogen-purged 30-gallon stainless steel reactor. Additional NMP (13.62 kg) is added to the reactor along with canola methyl esters (54.48 kg) comprising by weight 4.5 percent methyl palmitate, 2.9 percent methyl stearate and other saturates, 62.2 percent methyl oleate and other mono-unsaturated methyl esters, 20.4 percent methyl linoleate, and 9.0 percent methyl linolenate. The reactor is then heated to 90° C. under 400 psig (2,758 kPa) pressure of synthesis gas (1:1 hydrogen:carbon monoxide) with mixing via mechanical agitation at 250 rpm. The reactor pressure is maintained at 400 psig (2,758 kPa) by the addition of fresh synthesis gas for 4.5 hours. An aldehyde product (59.9 kg) is isolated by removing the catalyst solution through aqueous extraction as described in U.S. Pat. No. 5,180,854, incorporated herein by reference, wherein water is added to the crude hydroformylation product fluid to obtain by phase separation a nonpolar phase containing an aldehyde product comprising a plurality of formyl-substituted fatty acid esters and a polar phase comprising NMP, water, the rhodium-ligand complex catalyst, and free dicyclohexyl-(3-sulfonoylphenyl)phosphine mono-sodium salt ligand. The composition of the aldehyde product in terms of percent monoals, di-als, tri-als, and impurities (lactols, cyclic ethers, lactones, dimers) is set forth in Table 1. The average functionality number (AFN) of the aldehyde is 1.01 and the di-al/tri-al weight ratio is 3.05/1.

TABLE 1
Aldehyde Compositions Derived from Hydroformylation
of Canola Methyl Esters

| | | Example # | | | |
|---|---|---|---|---|---|
| | | 1 | | 2 | |
| Components | MW⁵ | Wt % | Mol % | Wt % | Mol % |
| Methyl stearate¹ | 296 | 17.86 | 19.66 | 9.83 | 10.96 |
| Methyl palmitate | 270 | 4.14 | 5.03 | 4.14 | 5.07 |
| Mono-als² | 326 | 54.02 | 54.04 | 56.38 | 57.08 |
| Di-als³ | 354 | 17.38 | 15.89 | 20.98 | 19.56 |
| Tri-als | 382 | 5.70 | 4.80 | 7.53 | 6.51 |
| Lactols (Cyclic ethers)⁴ | 356 | 0.33 | 0.30 | 0.60 | 0.56 |
| Lactones | 354 | 0.00 | 0.00 | 0.00 | 0.00 |
| Dimers | 656 | 0.57 | 0.29 | 0.54 | 0.27 |
| Total | | 100.00 | 100.00 | 100.00 | 100.00 |
| Di-als/Tri-als | | 3.05 | | 2.79 | |
| AFN | | | 1.01 | | 1.17 |
| Conversion % | | 85.0 | | 93.0 | |

¹Including unconverted unsaturated fatty acid methyl esters as the major components.
²Including both saturated and unsaturated mono-aldehydes, Also included are small amounts (0.1 to 1.5 wt %) of mono-aldehydes having C₁₆ and C₂₀ chains.
³Including both saturated and unsaturated di-aldehydes.
⁴Cyclic ethers are believed to be formed during GC analysis by dehydration of the lactols.
⁵Average molecular weight where more than one component is lumped together based on functionality.

### Example 2

The hydroformylation of canola methyl esters is repeated as described in Example 1 to prepare a second aldehyde sample, with the exception that the process is run for a longer time to a conversion of 93 percent to achieve an aldehyde having an AFN of 1.17 and a di-al/tri-al weight ratio of 2.79/1. Refer to Table 1.

### Examples 3 to 5

Alcohol Monomer 1 (Example 3): An up-flow tubular reactor is packed with a commercial supported nickel catalyst (440 mL, Sud-Chemie C46-8-03). The inlet of the reactor is comprised of two liquid feeds and one gas feed that are joined before entering the reactor. The two liquid feeds consist of the hydroformylated canola methyl ester of Example 1 hereinabove and recycled hydrogenation product from the same aldehyde supply. The flow rate of the hydroformylated canola methyl ester is 5 g/min; the flow rate of the recycled hydrogenation product is 19 g/min. Total Liquid Hourly Space Velocity is 3.51 hr⁻¹. Hydrogen gas is fed to the reactor at 2,000 standard cubic centimeters per minute (Gas Hourly Space Velocity 272 hr¹), and the reactor is heated to 143° C. Pressure is set at 830 psig (5,723 kPa). Analysis of the mixture after hydrogenation yields the alcohol composition Monomer 1 described in Table 2.

Alcohol Monomer 3 (Example 5) is produced similarly, with the exception that the aldehyde product of Example 2 replaces the aldehyde product of Example 1 as the feed to the hydrogenation. The composition of Alcohol Monomer 3 is set forth in Table 2.

Alcohol Monomer 2 (Example 4) is produced by mixing alcohol Monomer 1 and alcohol Monomer 3 in a 57:43 weight ratio with the results set forth in Table 2.

TABLE 2
Alcohol Compositions¹

| | | Example # | | | | | |
|---|---|---|---|---|---|---|---|
| | | 3 | | 4 | | 5 | |
| | | Alcohol Monomer # | | | | | |
| | | 1 | | 2 | | 3 | |
| Components | MW⁵ | Wt % | Mol % | Wt % | Mol % | Wt % | Mol % |
| Methyl | 298 | 19.31 | 21.25 | 15.76 | 17.50 | 11.05 | 12.42 |
| stearate | | | | | | | |
| Methyl | 270 | 5.55 | 6.74 | 5.59 | 6.85 | 5.64 | 7.00 |
| Palmitate² | | | | | | | |
| Monols³ | 328 | 51.10 | 51.08 | 51.21 | 51.66 | 51.35 | 52.46 |
| Diols | 358 | 13.03 | 11.93 | 14.82 | 13.70 | 17.19 | 16.09 |
| Triols | 388 | 4.61 | 3.90 | 5.60 | 4.77 | 6.90 | 5.96 |
| Lactols | 356 | 1.99 | 1.83 | 2.15 | 2.00 | 2.36 | 2.22 |
| (Cyclic | | | | | | | |
| ethers)⁴ | | | | | | | |
| Lactones | 354 | 2.52 | 2.33 | 2.46 | 2.30 | 2.37 | 2.25 |
| Dimers | 656 | 1.88 | 0.94 | 2.41 | 1.22 | 3.12 | 1.59 |
| Total | | 99.99 | 100.00 | 100.00 | 100.00 | 99.98 | 100.00 |
| Diols/Triols | | 2.82 | | 2.65 | | 2.49 | |
| AFN | | | 0.93 | | 1.00 | | 1.10 |

¹Unsaturates are not detected in alcohol Monomers 1 and 3, from which alcohol Monomer 2 is also prepared.
²Includes C₁₄ and C₂₀ saturates.
³Includes small amounts (0.1 to 1.5 wt %) of mono-alcohols having C₁₆ and C₂₀ chains.
⁴Cyclic ethers are believed be formed during GC analysis by dehydration of the lactols.
⁵Average molecular weight where more than one component is lumped together based on functionality.

### Examples 6 to 8

### Preparation of Polyols

### Example 6

Alcohol Monomer 1 (39000 g), prepared hereinabove in Example 3, is charged to a 30 gallon stainless steel jacketed reactor vessel together with a trifunctional poly(ethylene oxide) (Voranol™ IP 625 brand, The Dow Chemical Company; 17515 g; approx. 620 molecular weight). The reactor vessel is equipped with a nitrogen sparger, a turbine for gas dispersion, a vacuum system, and hot oil as a heating medium. The mixture is devolatilized by heating to 150° C. under 500 mmHg (66.7 kPa) and a nitrogen flow (1.0 standard cubic feet per minute, scfm). The speed of the agitator is set at 200 rpm. Tin ethylhexanoate (28.26 g) is added, and the reaction mixture is heated to 195° C. under atmospheric pressure and a nitrogen flow of 1.2 scfm. The pressure is reduced to 500 mmHg (66.7 kPa) and the reaction is continued for another 1.5 hrs. A polyol having a hydroxyl number of 81.8 and a viscosity of 1610 centipoise (cP) at 25° C. is obtained. ASTM 4274 is used to determine hydroxyl number. ASTM D4878 is used to determine viscosity.

### Example 7

The procedure of Example 6 is repeated using Alcohol Monomer 2, prepared as a mixture of Alcohol Monomer 1 (22230 g; 57 wt. percent) and Alcohol Monomer 3 (16770 g; 43 wt percent) (prepared in Examples 3 and 5 hereinabove), Voranol IP-625 (17350 g), and tin catalyst (28.18 g). A polyol having a hydroxyl number of 91.8 and a viscosity of 1650 cP at 25° C. is obtained.

### Example 8

The procedure of Example 6 is repeated using Alcohol Monomer 3 (39000 g, prepared as in Example 5), Voranol IP-625 (17133 g), and tin catalyst (28.07 g). A polyol having a hydroxyl number of 99.5 and a viscosity of 2290 cP at 25° C. is obtained.

### Examples 9-17

### Preparation of Urethane Flexible Foams

A series of flexible polyurethane foams are prepared using the polyols prepared in Examples 6 to 8. The components of the foam formulation include the following. Each foam is prepared individually by metering all of the components and additives indicated in Table 3 of a given formulation except for the catalysts, and weighing them into a one quart (0.965 liter) capacity metal cup. Component temperatures are approximately 20-30° C. In each case, 50 parts of a copolyester canola oil-based polyol is added with 50 parts of a nominally trifunctional, 1200 equivalent weight random copolymer of 87 percent propylene oxide and 13 percent ethylene oxide, by weight, (commercially available from The Dow Chemical Company as Voranol® 3512 brand polyol). The contents are premixed for 15 seconds at 1800 rpm using a high shear mixer capable of mixing speeds of 3,000 rpm. A tin catalyst indicated in Table 3, which is dispensed by weight, is added to the stirred components and mixed for an additional 15 seconds at 1800 rpm. A sufficient quantity of an 80/20 mixture of the 2,4- and 2,6-isomers of toluene diisocyanate is added to the mixture to provide an isocyanate index of 102, and the resulting mixture is mixed vigorously for 3 seconds at 2,400 rpm. The cup contents are then poured into a 38×38×25 cm (15×15×10 inch) wooden box lined with a polyethylene bag. The blow off time and any other distinct reaction characteristics are visually observed. The resulting foam buns are allowed to cure overnight under a ventilated fume hood, after which they are placed in ambient storage for a period of 7 days before being submitted for physical property assessment. ASTM test method designation D 3574-03 is used for evaluating the physical properties of the foam. Three foams are made from each polyol. Foam results are as indicated in Table 4 below.

TABLE 3
Formulation for Preparing Polyurethane Foams

| | Amount |
|---|---|
| Components and additives | (parts by wt) |
| Voranol ™ brand 3512 polyol | 50 |
| (The Dow Chemical Company) | |
| Canola natural oil polyol | 50 |
| Water | 4.5 |
| Amine catalyst Dabco 8264 | 0.15 |
| (Air Products & Chemicals) | |
| Silicone surfactant Naix ™ L620 | 0.60 |
| (GE) | |
| Stannous octoate Niax ® T-9 | Variable |
| | (per Table 4) |
| Toluene diisocyanate (T-80 Index) | 102.00 |

TABLE 4
Properties of Foams made from Canola Natural Oil Polyols¹

| | Example | | | | | | | | |
|---|---|---|---|---|---|---|---|---|---|
| | 9 | 10 | 11 | 12 | 13 | 14 | 15 | 16 | 17 |
| | Alcohol Monomer | | | | | | | | |
| | 1 | | | 2 | | | 3 | | |
| Monomer AFN | 0.93 | 0.93 | 0.93 | 1.01 | 1.01 | 1.01 | 1.10 | 1.10 | 1.10 |
| Polyol OH (hydroxyl | 82 | 82 | 82 | 92 | 92 | 92 | 100 | 100 | 100 |
| no.) | | | | | | | | | |
| Dabco T-9 (pphp) | 0.08 | 0.11 | 0.14 | 0.08 | 0.11 | 0.14 | 0.08 | 0.11 | 0.14 |
| Comments | Has | Has | | Has | | | | | |
| | splits | splits | | splits | | | | | |
| Airflow (ft³/min) | 7.2 | 6.6 | 6.4 | 6.4 | 5.9 | 4.7 | 5.3 | 3.4 | 1.6 |
| Compression set (%) | 14.7 | 21.5 | 32.6 | 14.9 | 23.1 | 20.8 | 14.8 | 19.1 | 48.2 |
| Density (lb/cu_ft) | 1.54 | 1.58 | 1.51 | 1.54 | 1.51 | 1.53 | 1.61 | 1.61 | 1.51 |
| 25% IFD (lbf) | 24.1 | 30.9 | 30.6 | 29.4 | 33.4 | 37.3 | 39.5 | 42.6 | 45.4 |
| 65% IFD (lbf) | 55.1 | 66.2 | 62.1 | 62.1 | 67.8 | 76.2 | 81.5 | 87.9 | 94.1 |
| 25% Return (lbf) | 15.3 | 19.7 | 18.1 | 18.6 | 20.1 | 22.6 | 24.4 | 26.1 | 25.8 |
| Support Factor (%) | 2.3 | 2.1 | 2 | 2.1 | 2 | 2 | 2.1 | 2.1 | 2.1 |
| Hysteresis (%) | 63 | 64 | 59 | 63 | 60 | 61 | 62 | 61 | 57 |
| Resiliency (%) | 31 | 29 | 28 | 32 | 30 | 32 | 33 | 33 | 31 |
| Tear Strength (lbf/in) | 1.97 | 2.08 | 2.05 | 2.14 | 2.09 | 2.19 | 1.92 | 2.12 | 1.9 |
| Tensile Strength (psi) | 10 | 11.3 | 12.7 | 12.6 | 14.5 | 15.4 | 16.6 | 17.2 | 17.9 |
| Elongation (%) | 83 | 88 | 126 | 96 | 115 | 108 | 103 | 104 | 103 |

¹50/50 Voranol 3512 polyol/canola natural oil polyol; 4.5 parts water; 0.15 parts Dabco 8264, 0.6 parts L620 surfactant, 102 parts TDI (80 Index)

From Table 4 it is seen that commercially acceptable flexible urethane foams are prepared from canola ester-based polyols. In particular, when the polyol has an AFN of 0.93, a catalyst (T-9) concentration of 0.14 percent is preferred. Below this catalyst concentration, airflow and other foam properties are acceptable, but the foam has splits. Using a polyol having an AFN of 1.01, commercially acceptable flexible foams are prepared at T-9 catalyst concentrations of 0.11 and 0.14 parts by weight. Using a polyol having an AFN of 1.10, acceptable foams are prepared at catalyst concentrations of 0.08 and 0.11 percent. At a concentration of 0.14 percent, the foam shows a reduced air flow.

### Comparative Experiment 1

A comparative monomer alcohol composition having an average functionality number 0.33 is synthesized and used to prepare a polyol from which a polyurethane is made for comparison with the polyurethanes of the invention.

At the start, a soy-based alcohol composition is prepared by hydroformylating a mixture of soy methyl esters in a manner similar to Example 1 hereinabove to obtain a soy-based aldehyde composition, which is hydrogenated in a manner similar to Example 3 hereinabove. The resulting soy-based monomer alcohol has composition CE-1A and an average functionality number of 1.12, as shown in Table 5. The monomer alcohol composition is fed into short-path evaporator (SPE), and a first distillate obtained therefrom is reprocessed in a second pass through the SPE to obtain a second distillate shown in Table 5 as composition CE-1B having an average functionality number 0.33, details as follows.

A first distillate sample is prepared by feeding degassed soy-based alcohol composition CE-1A at a rate of 10-20 g/min to a Pope 4″ SPE operating at a vacuum of 0.08-0.11 mm Hg (11-15 Pa) and 480 RPM. The SPE jacket temperature and internal condensing coil are maintained at 180° C. and 35° C. The equipment is operated such that a residue to feed fraction of 0.3-0.5 is obtained. The first distillate is reprocessed in the SPE such that a feed rate of 110-155 g/min resulted in a residue to feed ratio of 0.55-0.75. The SPE jacket, condensing coil, RPM and vacuum are maintained at 180° C., 37° C., 480 RPM, and 0.16-0.21 mm Hg (21-28 Pa), respectively.

A second distillate is prepared by feeding degassed soy-based alcohol composition at a rate of 12-25 g/min to the Pope 4″ SPE operating at a vacuum of 0.09-0.2 mm Hg (12-27 Pa) and 480 RPM. The SPE jacket temperature and internal condensing coil are maintained at 180° C. and 35-45° C. The equipment operated such that a residue to feed fraction of 0.32-0.45 is obtained in three separate runs. The distillate is reprocessed in the SPE such that a feed rate of 70-80 g/min resulted in a residue to feed ratio of 0.48-0.50. The SPE jacket, condensing coil, RPM and vacuum are maintained at 180° C., 45° C., 480 rpm and 0.06-0.12 mm Hg (8.0−1.60 kPa), respectively.

Blending the first and second distillates in a 55:45 weight ratio, respectively, gives the alcohol monomer CE-1B having an average functionality number of 0.33 shown in Table 5.

Another alcohol monomer blend is prepared by mixing alcohol monomer CE-1B with Alcohol Monomer 3 of the invention, prepared hereinabove, in a weight ratio of 80 percent to 20 percent, respectively, by weight. The resulting blended alcohol monomer composition has an average functionality number of 0.80, shown as Alcohol Monomer CE-1C in Table 5.

TABLE 5
Comparative Monomers CE-1A and CE-1B

| | Alcohol Monomer No. | | |
|---|---|---|---|
| | | | CE-1C |
| | | | (Blend of |
| | CE-1A | CE-1B | Monomers 3 and |
| | (Soy Alcohol) | (Distillates) | CE-1B |
| AFN | 1.12 | 0.33 | 0.80 |
| Diol/Triol Ratio | 15.49 | 36.60 | 2.92 |
| Monomer | | | |
| normalized | | | |
| composition | Wt. % | Wt. % | Wt. % |
| Methyl Stearate | 15.57 | 40.90 | 23.63 |
| Methyl Palmitate | 9.41 | 24.32 | 9.30 |
| Monols | 35.7 | 31.94 | 47.27 |
| Diols | 27.72 | 1.83 | 10.79 |
| Triols | 1.79 | 0.05 | 3.70 |
| Lactols/Cyclic | 1.59 | 0.79 | 1.75 |
| ethers | | | |
| Lactones | 1.31 | 0.05 | 2.03 |
| Dimers | 6.19 | 0.12 | 1.53 |
| Others | 0.72 | — | — |
| Total | 100.0 | 100.00 | 100.00 |

The alcohol monomer composition CE-1C having a diol/triol ratio of 2.92 and an average functionality number of 0.80 is used to prepare a polyol in the manner described in Example 6 hereinabove. Specifically, 3044.16 g of the blended alcohol CE-1C are reacted with 1396.93 g of IP 625 in the presence of 2.22 g tin catalyst to yield a polyol having a hydroxyl number of 66.1 and a viscosity of 1160 centipoise at 25° C. The polyol is used to prepare a polyurethane in the manner described in Example 9 hereinabove, with the results shown in Table 6, from which it is seen that the comparative polyurethanes prepared with a polyol derived from a monomer alcohol having an average functionality number 0.80 are unacceptable for use in any application including flexible foams. The comparative foam collapses and its properties are not measurable.

TABLE 6
Properties of Foams made from Comparative Polyol¹

| Example | CE-1B | | |
|---|---|---|---|
| Monomer AFN | 0.80 | 0.80 | 0.80 |
| Polyol OH (hydroxyl | 66.1 | 66.1 | 66.1 |
| no.) | | | |
| Dabco T-9 (pphp) | 0.08 | 0.11 | 0.14 |
| Comments | Foam | Foam | Foam |
| | Collapsed | Collapsed | Collapsed |
| Airflow (ft³/min) | N.M. | N.M. | N.M. |
| Compression set (%) | N.M. | N.M. | N.M. |
| Density (lb/cu_ft) | N.M. | N.M. | N.M. |
| 25% IFD (lbf) | N.M. | N.M. | N.M. |
| 65% IFD (lbf) | N.M. | N.M. | N.M. |
| 25% Return(lbf) | N.M. | N.M. | N.M. |
| Support Factor (%) | N.M. | N.M. | N.M. |
| Hysteresis (%) | N.M. | N.M. | N.M. |
| Resiliency (%) | N.M. | N.M. | N.M. |
| Tear Strength (lbf/in) | N.M. | N.M. | N.M. |
| Tensile Strength (psi) | N.M. | N.M. | N.M. |
| Elongation (%) | N.M. | N.M. | N.M. |

¹50/50 Voranol 3512 polyol/natural oil polyol; 4.5 parts water; 0.15 parts Dabco 8264, 0.6 parts L620 surfactant, 102 parts TDI (80 Index)
²N.M. = not measurable.

### Comparative Experiment 2

Alcohol Monomer 3, prepared in Example 5 hereinabove, is degassed for over 48 hours. The monomer is fed at a rate of 65-75 g/min to a jacketed Pope 4″ short-path evaporator (SPE). The SPE jacket temperature and condenser coil are maintained at 200° C. and 38° C., respectively. The SPE pressure is maintained at 0.11-0.17 mm Hg (16-23 Pa). The process is operated such that a residue to feed ratio of 0.4-0.5 is achieved with wiper blades rotating at 480 rpm. A residue is recovered having the composition shown as CE-2A in Table 7.

An alcohol monomer blend is prepared by mixing the alcohol monomer CE-2A with Alcohol Monomer 3 in a ratio of 40 percent to 60 percent, by weight, respectively. The resulting blend has a diol/triol ratio of 2.65 and an average functionality number of 1.30, shown as composition CE-2B in Table 7.

TABLE 7
Comparative Monomers CE-2A and CE-2B

Monomer No.

| | | CE-2B (Blend of |
|---|---|---|
| | | Monomers 3 and |
| | CE-2A | CE-2A |
| AFN | 1.61 | 1.30 |
| Diol/Triol Ratio | 2.77 | 2.65 |
| Monomer | | |
| normalized | | |
| composition | Wt. % | Wt. % |
| Methyl Stearate | 0.92 | 7.00 |
| Methyl Palmitate | 0.13 | 3.44 |
| Monols | 43.82 | 48.34 |
| Diols | 36.33 | 24.85 |
| Triols | 13.12 | 9.39 |
| Lactols/Cyclic | 0.93 | 1.79 |
| ethers | | |
| Lactones | 0.24 | 1.52 |
| Dimers | 4.51 | 3.68 |
| Total | 100.00 | 100.00 |

The alcohol monomer composition CE-2B having an average functionality number of 1.30 is used to prepare a polyol in the manner described in Example 6 hereinabove. In particular, 4510.50 g blend CE-2B, 1947.41 g IP-625, and 3.23 g tin catalyst are used yielding a polyol having a hydroxyl number of 112.7 and a viscosity of 4510 centipoise at 25° C. The polyol is used to prepare a polyurethane in the manner described in Example 9 hereinabove. Results of the testing of the polyurethane are shown in Table 8.

TABLE 8
Properties of Foams made from Comparative Polyol¹

| Example | CE-2B | | |
|---|---|---|---|
| Monomer AFN | 1.3 | 1.3 | 1.3 |
| Polyol OH (hydroxyl | 112.7 | 112.7 | 112.7 |
| no.) | | | |
| Dabco T-9 (pphp) | 0.08 | 0.11 | 0.14 |
| Comments | — | — | — |
| Airflow (ft³/min) | 1.6 | 0.3 | 0.1 |
| Compression set (%) | 20.5 | 20.2 | 64.6 |
| Density (lb/cu_ft) | 1.578 | 1.548 | 1.524 |
| 25% IFD (lbf) | 49.9 | 52.4 | 52.7 |
| 65% IFD (lbf) | 101.2 | 108.5 | 112.8 |
| 25% Return(lbf) | 30.2 | 30.2 | 28.7 |
| Support Factor (%) | 2.0 | 2.1 | 2.1 |
| Hysteresis (%) | 61 | 58 | 55 |
| Resiliency (%) | 33 | 28 | 22 |
| Tear Strength (lbf/in) | 1.8 | 1.5 | 1.5 |
| Tensile Strength (psi) | 20.2 | 18.5 | 18.7 |
| Elongation (%) | 97 | 83 | 82 |

¹50/50 Voranol 3512 polyol/natural oil polyol; 4.5 parts water; 0.15 parts Dabco 8264, 0.6 parts L620 surfactant, 102 parts TDI (80 Index)

From Table 8 it is seen that the polyurethane foams prepared with comparative blend CE-2B having an average functionality number of 1.30 are not suitable for flexible foam applications. In particular, the air flows are very low ranging only from 0.1 to 1.6 ft³/min. In contrast, the foams prepared in accordance with the invention, having an average functionality number between 0.90 and 1.20 are suitable for flexible foam applications, with significantly higher air flows up to 7.2 ft³/min.

What is claimed is:

1. An aldehyde composition comprising a mixture of formyl-substituted fatty acids and/or fatty acid esters comprising in terms of formyl distribution from greater than about 30 to less than about 95 percent mono-aldehyde, from greater than about 0.4 to less than about 37 percent di-aldehyde, and from greater than about 0.1 to less than about 34 percent tri-aldehyde, and further comprising from greater than about 3 to less than about 30 percent saturates and from greater than about 1 to less than about 20 percent unsaturates, by weight, based on the total weight of the composition, and further having a di-al/tri-al weight ratio of less than 5/1 and an average functionality number ranging from greater than 0.96 to less than 1.26.

2. The aldehyde composition of claim 1 wherein the di-al/tri-al weight ratio is less than 4.5/1.

3. The aldehyde composition of claim 1 comprising less than about 10 weight percent total impurities.

4. The aldehyde composition of claim 1 comprising from greater than about 50 to less than about 90 percent monoaldehyde, from greater than about 2 to less than about 27 percent di-aldehyde, and from greater than about 0.6 to less than about 23 percent tri-aldehyde, by weight.

5. The composition of claim 1 wherein the composition is prepared by hydroformylating a mixture of unsaturated fatty acids or unsaturated fatty acid esters obtained from a seed oil in the presence of carbon monoxide and hydrogen and a hydroformylation catalyst under reaction conditions.

6. The composition of claim 5 wherein the seed oil is selected from naturally occurring and genetically modified seed oils and mixtures of such seed oils comprising in terms of fatty acid chains from greater than about 50 to less than about 90 percent mono-unsaturated fatty acids; from greater than about 1 to less than about 45 percent di-unsaturated fatty acids; and from greater than about 0.4 to less than about 45 percent tri-unsaturated fatty acids, by weight, and further having a weight ratio of di-unsaturates to tri-unsaturates less than about 3:1.

7. An alcohol composition comprising a mixture of hydroxymethyl-substituted fatty acids and/or fatty acid esters comprising in terms of hydroxy distribution from greater than about 30 to less than about 90 percent mono alcohol, from greater than about 0.4 to less than about 34 percent di-alcohol, and from greater than about 0.1 to less than about 31 percent tri-alcohol, and further comprising from greater than about 3 to less than about 35 percent saturates and less than about 10 percent unsaturates, by weight, based on the total weight of the composition, and further having a diol/triol weight ratio less than 5/1 and an average functionality number ranging from greater than 0.90 to less than 1.20.

8. The alcohol composition of claim 7 having a diol/triol weight ratio of less than 4.5/1.

9. The alcohol composition of claim 7 comprising less than about 10 weight percent total impurities selected from the group consisting of lactols, lactones, saturated cyclic ethers, unsaturated cyclic ethers, and heavies.

10. The alcohol composition of claim 7 comprising from greater than about 50 to less than about 86 percent monoalcohol, from greater than about 2 to less than about 24 percent diol, and from greater than about 0.6 to less than about 20 percent triol, by weight.

11. The composition of claim 7 prepared by hydroformylating a mixture of unsaturated fatty acids or unsaturated fatty acid esters obtained from a seed oil in the presence of carbon monoxide and hydrogen and a hydroformylation catalyst under reaction conditions sufficient to prepare a mixture of formyl-substituted fatty acids or fatty acid esters; and thereafter hydrogenating the mixture of formyl-substituted fatty acids or fatty acid esters with hydrogen in the presence of a hydrogenation catalyst under reaction conditions sufficient to form the alcohol composition.

12. The composition of claim 11 wherein the seed oil is selected from naturally occurring and genetically modified seed oils and mixtures of such seed oils comprising in terms of fatty acid chains from greater than about 50 to less than about 90 percent mono-unsaturated fatty acids; from greater than about 1 to less than about 45 percent di-unsaturated fatty acids; and from greater than about 0.4 to less than about 45 percent tri-unsaturated fatty acids, by weight, and further having a weight ratio of di-unsaturates to tri-unsaturates less than about 3:1.

13. The composition of claim 7 being prepared by mixing together two or more alcohol compositions selected from the group consisting of alcohol compositions having an average functionality number from greater than 0.90 to less than 1.20 and one or more alcohol compositions each having an average functionality number less than 0.90 or greater than 1.20.

14. A polyester polyol composition comprising a reaction product of an alcohol composition of claim 7 with an initiator compound having from 2 to 8 hydroxyl groups per molecule and a molecular weight of about 90 to about 6000.

15. A polyurethane comprising a reaction product of a polyol composition that includes the polyester polyol composition of claim 14 with at least one polyisocyanate.

16. The polyurethane of claim 15 which is a flexible foam.
